(19) United States
(12) Patent Application Publication
(10) Pub. No.: US 2022/0118438 A1
(43) Pub. Date: Apr. 21, 2022

(54) METHOD FOR ADJUSTING A CELL CONCENTRATION AND/OR PARTICLE CONCENTRATION IN A DISPENSING SYSTEM

(71) Applicant: cytena GmbH, Freiburg (DE)

(72) Inventor: Jonas SCHÖNDUBE, Freiburg (DE)

(21) Appl. No.: 17/417,448

(22) PCT Filed: Dec. 11, 2019
(86) PCT No.: PCT/EP2019/084594

§ 371 Date: Jun. 23, 2021

(30) Foreign Application Priority Data
Dec. 27, 2018 (LU) LU101085

(51) Int. Cl. B01L 3/02

(52) U.S. Cl. CPC B01L 3/0293; B01L 3/0237; B01L 2400/0487; B01L 2400/0436; B01L 2300/14; B01L 2200/0652; B01L 2200/143; B01L 2300/0663

(57) ABSTRACT

The invention relates to a method for setting a cell concentration and/or a particle concentration in a dispensing device that has a fluid chamber into which a liquid sample is introduced that has a liquid and cells and/or particles, wherein the cell concentration and/or the particle concentration is determined, in particular in one region of the dispensing device, and the cell concentration and/or particle concentration that has been determined is compared with a target value and the cells and/or particles, in particular one or the other, that are present in the fluid chamber are moved or not moved as a function of the result of the comparison.

### CROSS-REFERENCE TO RELATED APPLICATIONS

The present application is the U.S. national phase of International Application No. PCT/EP2019/084594 filed Dec. 11, 2019, which claims the benefit of and priority to Luxembourgian Patent Application No. 101085 filed Dec. 27, 2018, the entire disclosure of which is incorporated herein by reference.

### FIELD

The disclosure relates to a method for adjusting a cell concentration and/or particle concentration in a dispensing device. In addition, the disclosure relates to a dispensing apparatus. Moreover, the disclosure relates to a computer program, a data carrier on which the computer program is stored and a data carrier signal that the computer program transmits.

### BACKGROUND

A plurality of apparatuses are known from the prior art, by means of which a liquid sample that has a liquid and at least one cell can be discharged. Apparatuses are known in the art in which the discharge of the liquid is accomplished by means of free jet pressure methods. A distinction is made between such apparatuses in which the discharge of the liquid is accomplished by a drop-on-demand methodology or a continuous-jet methodology. In the drop-on-demand methodology, individual drops are deliberately generated from a dispensing device of the apparatus at a selected time. Thus, individual drops are generated on command using a separate activation signal.

In contrast to the drop-on-demand methodology, in the continuous-jet methodology, a thin liquid jet is dispensed from the dispensing apparatus by pressure, and after discharge from the dispensing apparatus, the liquid jet breaks up into individual drops that can be diverted electrostatically. In the continuous jet methodology, a separate activation signal is thus not furnished for each single drop, and the individual drops cannot be deliberately generated at a selected time.

In apparatuses that are known from the prior art, the problem often arises that the cell concentration in the liquid sample is too high, such that liquid drops are discharged that have more than one cell, which are unusable for further processing. The problem may also arise that the cell concentration in the liquid sample is too low so that a plurality of liquid drops that do not have any cells must be discharged in succession such that the process for discharging the liquid drops is not efficient.

### SUMMARY

The object of the disclosure consists of providing an improved method.

The object is accomplished by a method for setting a cell concentration and/or a particle concentration in a dispensing device that has a fluid chamber into which a liquid sample is introduced that has a liquid and cells and/or particles, wherein the cell concentration and/or the particle concentration is determined, in particular in one region of the dispensing device, and the cell concentration and/or particle concentration that has been determined is compared with a target value and the cells and/or particles, in particular one or the other, that are present in the fluid chamber are moved or not moved as a function of the result of the comparison.

A further object of the disclosure consists of providing an improved dispensing apparatus.

This object is accomplished by a dispensing apparatus that carries out a method according to the disclosure.

The object is also accomplished by a dispensing apparatus having a control apparatus, a dispensing device with a fluid chamber for accommodating a liquid sample that has a liquid and cells and/or particles, and an evaluation apparatus for determining the cell concentration and/or particle concentration; wherein the control apparatus for setting a cell concentration and/or particle concentration in the dispensing device causes or does not cause a movement of the cells and/or particles present in the fluid chamber, as a function of a result comparing the cell concentration and/or particle concentration that has been determined, and in particular one or the other, with a target value.

The solution according to the disclosure has the advantage that the cell concentration and/or the particle concentration can be adjusted actively. Adjusting the cell concentration and/or the particle concentration is straightforwardly possible as a result of the cells and/or particles that are present in the fluid chamber being either moved or not moved, in particular one or the other. As a result the cell concentration and/or particle concentration can be straightforwardly adjusted.

In particular, the number of liquid drops that have multiple cells and/or particles can be reduced. In addition, the method can also be carried out more efficiently because the amount of liquid that does not contain a cell and/or particles, in particular the number of such liquid drops, is reduced. Thus, the method can be performed in such a way that after a maximum of 50 dispensing operations in which the respective discharged liquid sample has no cell and/or no particle, a dispensing operation is carried out in which the liquid sample has a cell and/or a particle.

For the purpose of the disclosure, the discharged liquid sample can be a drop of liquid, in particular free-flying. Alternatively, the discharged liquid sample may be a liquid jet, which may break up into individual liquid drops optionally after being discharged from the liquid discharge device. The liquid drop can have a volume in a range between 10 pl (picolitres) and 50 nl (nanolitres). Moreover, the discharged liquid drop can have one cell, in particular a single cell, and/or a particle, in particular a single particle.

The liquid of the liquid sample can have a composition that is conducive to cell growth. The particle can be a glass or polymer bead and have a substantially similar volume to the cell. The cell is a biological cell, in particular the cell is the smallest unit of life that is autonomously capable of reproduction and self-preservation.

Depending on the result of the comparison, the cells and/or particles can be moved from a rest state. However, it is also possible that cells and/or particles that have already been moved are moved, depending on the result of the comparison. The type of movement of the cells and/or particles changes in this case. Thus, the cells and/or particles can be moved in such a way that, for example, their speed and/or direction of movement is changed compared to the state before determining the cell concentration and/or particle concentration.

In this case, the cells and/or particles can be moved as a result of moving the liquid sample. When the liquid sample present in the fluid chamber is moved, in particular, the arrangement of the cells and/or particles arranged in the liquid is changed. In this case, the more the liquid sample is set in motion, the more the arrangement of the cells and/or particles changes.

Alternatively, it is possible for exclusively the cells and/or particles to be moved, for example by applying a sound field, namely in particular by acoustophoresis. In this case, the liquid is not set in motion. Other examples of means by which cells and/or particles can be moved are electrophoresis, magnetophoresis, optofluidics or hydrodynamics. Any combination of the above possibilities may also be used.

Alternatively, the sound field and/or the other above-mentioned examples can be used to hold the cells and/or particles in their current position, thus not moving them.

As a result of the movement of the liquid present in the fluid chamber and thus the movement of the cells and/or particles in the liquid, a more homogeneous, or if needed a more inhomogeneous, distribution of the cells and/or particles in the liquid can be achieved. As a result, the cell concentration and/or particle concentration in the dispensing device can easily be adjusted.

The manner of movement, in particular the speed and/or direction of movement of the cells and/or particles, can be adjusted by appropriately controlling the dispensing device. In particular, by adjusting the intensity of, for example, the sound field and/or the flow rate or a pressure resulting from actuation, it is possible to adjust whether the cells and/or particles move, or how such movement takes place.

The target value can be a value that is or can be predetermined. In such a case, the target value can be entered by the user or determined automatically. The target value can be stored in an electrical memory. The target value can have a value in the range between 100 cells per millilitre and 10⁸ cells per millilitre.

The result of the comparison may be that the cell concentration and/or particle concentration is less than, or equal to, or greater than the target value.

The method can be carried out automatically. This signifies that the method, without user involvement, automatically adjusts the cell concentration and/or particle concentration.

In a particular embodiment, the cells and/or particles can be moved if the determined cell concentration and/or particle concentration is less than the target value. Moreover, the cells and/or particles can be moved differently, in particular less strongly, and preferably not at all, in a state in which the cell concentration and/or particle concentration is greater than or equal to the target value, compared to another state in which the cell concentration and/or particle concentration is less than the target value. This means that it is not strictly necessary for the cells and/or particles not to move if the cell concentration and/or particle concentration obtained is greater than the target value. “Less strong” in this case means that the speed of the cells and/or particles is less than in the other case.

In this case, if the determined cell concentration and/or particle concentration is greater than the target value or equal to the target value, it is possible that the cells and/or particles are not moved and/or are moved less and/or are moved otherwise. Moreover, in this case, at least one dispensing operation can be accomplished that lasts a predetermined time or in which a predetermined volume is dispensed. A liquid jet or drops can then be discharged. Alternatively or additionally, a predetermined number of dispensing operations can be carried out in this case. As a result, a predetermined number of liquid drops can be discharged.

In both cases, a large amount of liquid sample can be discharged in a short time in a straightforward manner. As a result, the cell concentration and/or particle concentration can be straightforwardly reduced. This effect can then occur, for example, if a region of the liquid sample with a high cell and/or particle concentration is ejected and the cells and/or particles settle in another region or the same region by sedimentation.

In this context, a method and/or a dispensing apparatus is particularly advantageous in which it is possible to selectively carry out one or the other of the above-mentioned modes of operation. The movement of the cells and/or particles can be caused by the control apparatus.

In this case, a dispensing operation can be carried out after a moving of the cells and/or particles. Alternatively or additionally, the dispensing operation can be carried out after a predetermined period of time from the time of determining the cell concentration and/or particle concentration. In the dispensing operation, liquid sample is discharged. After the dispensing operation, as will be described in greater detail below, the cell concentration and/or particle concentration in the observed region of the dispensing device may change in the desired direction.

The discharged liquid sample may not have any cells and/or any particles. Alternatively, the discharged liquid sample can have a single cell and/or a single particle. The discharged liquid sample can alternatively have more than one single cell and/or more than one single particle.

In a particular embodiment, to move the cells and/or particles in the fluid chamber, the liquid sample and/or the cells and/or particles can be mixed. This can be brought about by means of the control apparatus. As a result of the mixing of the liquid sample, a more homogeneous distribution, or if needed a more inhomogeneous distribution, of the cells and/or particles in the fluid chamber and/or within the liquid is realised. This is particularly advantageous because the cells and/or particles preferably collect on a bottom of the fluid chamber and are rearranged distributed within the fluid chamber due to the mixing of the liquid sample.

A more homogeneous or inhomogeneous distribution of the cells and/or particles in the fluid chamber, as the case may be, leads to an increase in the cell concentration and/or particle concentration in the region of the dispensing device, in particular in a discharge tubing of the dispensing device, after the dispensing operation by the dispensing device. This takes place because after the dispensing operation, liquid sample with a higher cell concentration and/or particle concentration flows from the fluid chamber into the discharge tubing.

As above-described, as a result of the mixing, the cells and/or particles are more homogeneously or inhomogeneously distributed in the liquid and/or in the fluid chamber, such that after the dispensing operation, the probability is greater that liquid sample flowing into the discharge tubing has more cells and/or particles than is the case in embodiments in which no mixing takes place of the liquid sample present in the fluid chamber. Mixing of the liquid sample refers to an operation in which the parts of the liquid sample are moved relative to each other in such a way that a new arrangement is created.

Mixing of the liquid sample in the fluid chamber can be straightforwardly accomplished by, alternatingly, a portion of the liquid in the fluid chamber being suctioned into a tubing and at least a portion of the liquid sample present in the tubing being discharged into the fluid chamber (reciprocal pumping). The alternating suctioning of the liquid into the tubing and discharge of the liquid from the tubing can be carried out multiple times in succession. As a result, the liquid sample remaining in the fluid chamber is particularly well mixed. Other ways to achieve movement of the liquid are: stirrer, propeller, magnetic stirrer, shaker or gassing.

The tubing can be immersed in the liquid sample and/or fluidically connected to a pressure or pump unit by means of which an underpressure or an overpressure can be reached in the tubing. After the overpressure has been generated and the liquid has been discharged, the tubing is vented. This ensures that the level of the liquid inside the tubing settles down to the level of the liquid sample in the fluid chamber. For venting, the tubing is fluidically connected to the environment.

The above-described mixing of the liquid sample by drawing in a portion of the liquid sample and discharging at least a portion of the liquid sample present in the tubing can be brought about by the control apparatus. It is advantageous that the mixing of the liquid sample can take place via a portion of the liquid sample present in the fluid chamber. Thus, no additional components and/or fluids are necessary in order to realise a mixing of the liquid sample. The portion of the liquid sample discharged from the tubing may be at least the portion of the liquid sample that was previously suctioned in.

The tubing can be removably reinserted into the dispensing device, in particular the fluid chamber, and/or can have a pipette shape. In particular, the tubing can be detachably reconnected to the dispensing device. This offers the advantage that the dispensing device can be swapped out after one use, which is often desired to avoid cross-contamination between two different liquid samples. The fluid chamber can be filled with the liquid sample, in particular for the first time, via the tubing.

In a particular embodiment, a portion of the liquid sample can be suctioned into the tubing. In this case, the suctioned-in portion of the liquid sample can be held in the tubing for a predetermined period of time. These process steps can be brought about by the control apparatus. Keeping the liquid sample in the tubing has the advantage of increasing the cell concentration and/or particle concentration of the liquid sample remaining in the fluid chamber. This takes place because cells and/or particles suctioned into the tubing can sediment over time and thus enter the fluid chamber from the tubing or accumulate in narrow regions, in particular the nozzle of the discharge tubing, and in extreme cases can clog these regions.

After the predetermined time has expired, another portion of the suctioned-in liquid sample, which is smaller than the suctioned-in portion of the liquid sample, can be discharged. As a result, a residual quantity of the suctioned-in liquid sample remains in the tubing after the discharge operation.

In this procedure, use is made of the fact that the cells and/or particles that have been suctioned into the tubing collect in a lower part of the tubing due to sedimentation. Thus, the liquid suctioned into the tubing has two different liquid regions with different cell concentrations and/or particle concentrations. In this case, one fluid region arranged closer to the bottom of the fluid chamber has a higher cell concentration and/or particle concentration than the other liquid region. The discharged other portion of the liquid may correspond to the liquid region with the higher cell concentration and/or particle concentration. By discharging exclusively the liquid region that has the higher cell concentration and/or particle concentration, the cell concentration and/or particle concentration of the liquid sample remaining in the fluid chamber is increased.

The above-described method can be carried out after the alternating suction of the liquid into the tubing and discharge of the liquid from the tubing. In this case, a thorough mixing of the liquid sample in the fluid chamber is realised, along with an increase in the cell concentration and/or particle concentration of the remaining liquid sample in the fluid chamber.

The above-described method, in particular the suctioning of the portion of the liquid sample into the tubing, can be carried out if the determined cell concentration and/or particle concentration is less than the target value.

In another particular embodiment, there is a delay for a certain period of time until the cells and/or particles have been concentrated, in particular by sedimentation, in a lower region of the fluid chamber. This lower region can be in fluidic contact with the discharge tubing so that the portion of the sample that is able to flow into the discharge tubing thus has a higher concentration. By slightly moving the cells and/or particles of the fluid chamber, it can be ensured that the cells and/or particles do not settle completely to the bottom of the fluid chamber.

In another particular embodiment, a larger quantity of sample is taken into the tubing and only a smaller quantity of sample is used for reciprocal pumping. The cells and/particles can sediment in the tubing in such a way that the upper part of the tubing contains a lower concentration and the lower part contains a higher concentration. Because only the smaller quantity of sample in the lower part of the tubing is used for mixing with the remaining sample in the fluid chamber, a higher particle and/or cell concentration can thus be achieved in the fluid chamber.

In a particular embodiment, the control apparatus can determine the cell concentration and/or particle concentration in the region of the dispensing device. In particular, the control apparatus may determine the cell concentration and/or particle concentration in the discharge tubing or a region of the discharge tubing or a region of the dispensing device spaced apart from the fluid chamber. The discharge tubing is the region of the dispensing device through which the liquid sample is passed shortly before discharge. The discharge tubing has a discharge opening through which the liquid sample is discharged from the dispensing device.

Alternatively or additionally, the cell concentration and/or particle concentration in the tubing and/or the liquid region that has the higher cell concentration and/or particle concentration in the tubing can be determined by means of the control apparatus. As a result, the time at which the liquid region with the higher cell concentration and/or particle concentration should be discharged can be straightforwardly ascertained.

In addition, by means of the control apparatus the volume of the liquid region having higher cell concentration and/or particle concentration that should be discharged from the tubing can also be determined.

The dispensing apparatus can be designed such that, in order to determine the cell and/or particle concentration, a portion of the liquid sample ejected from the dispensing device, in particular one or more liquid drops, is detected. In this case it is possible to detect whether the ejected portion of the liquid sample contains a cell and/or particles. In particular, the number of cells and/or particles contained in the ejected portion of the liquid sample can be detected. The dispensing apparatus in this case can have at least an imaging apparatus and/or an evaluation apparatus. The evaluation apparatus can be part of a computer.

By means of the imaging apparatus, at least one optical image of the discharged liquid sample, in particular of the liquid drop, can be generated. In addition, an optical image of the discharge tubing, or of a region of the discharge tubing of the dispensing device, can be generated. The region of the discharge tubing can correspond to an end region of the discharge tubing. In particular, the region under consideration may comprise a nozzle of the discharge tubing. The imaging apparatus can be a camera. The image can be used to determine the cell and/or particle concentration, as described in greater detail below.

In a particular embodiment, an image of an ejected portion of the liquid sample may be generated. This is useful if it is desired to determine the cell and/or particle concentration after the liquid sample has been discharged. The cell and/or particle concentration can be generated based on at least one image of the ejected liquid sample in flight. Alternatively or additionally, at least one image can be generated after the liquid sample has been applied to a surface, with the cell and/or particle concentration being generated based on the image. At least one image can also be generated of a container into which the ejected portion of the liquid sample is deposited.

Moreover, another image of the tubing can be generated by means of the imaging apparatus or a different imaging apparatus.

The evaluation apparatus can evaluate the generated image or the generated images. In particular, based on the generated image, it can be ascertained whether the liquid sample to be discharged, in particular the liquid drop to be discharged, or the discharged liquid sample, has a cell and/or particles. Alternatively or additionally, the evaluation apparatus can determine the number of cells and/or particles present in the discharge tubing and/or in the region of the discharge tubing based on the generated image.

The evaluation apparatus can also evaluate the other image. In particular, the evaluation apparatus can determine the number of cells and/or particles present in the tubing and/or determine the liquid region in the tubing that has the higher cell concentration and/or particle concentration.

The results obtained by the evaluation apparatus can be transmitted to the control apparatus. The control apparatus may have a processor and/or may be part of a computer. In addition, the control apparatus can be electrically connected to the evaluation apparatus. The control apparatus can be, in addition, electrically connected to the pressure unit, mixing unit or pump unit. In particular, depending on the result from the evaluation apparatus, the control apparatus can cause an underpressure or overpressure to be realised in the tubing by means of, in particular, the pressure unit or pump unit, in order to effect a mixing of the liquid sample present in the fluid chamber.

The control apparatus can straightforwardly determine the cell concentration and/or particle concentration via the ascertained number of cells and/or particles arranged in a discharge tubing of the dispensing device or in a region of a discharge tubing of the dispensing device. After the check to determine whether the cell concentration and/or particle concentration in the discharge tubing or region of the discharge tubing is too high or low, the above-described steps can be carried out to change the cell concentration and/or particle concentration in the discharge tubing or region of the discharge tubing. In addition, the control apparatus can determine the cell concentration and/or particle concentration in a tubing via the ascertained number of cells and/or particles arranged in the tubing.

Alternatively or additionally, in order to determine the cell concentration and/or the particle concentration, the control apparatus can determine the number of dispensed liquid drops that respectively have no cell and/or no particle, or a single cell and/or a single particle, or multiple cells and/or multiple particles.

The cell and/or particle concentration can be determined by determining the number of cells and/or particles per volume, or the number of cells or a volume ratio between the cell volume and the sample volume. Alternatively or additionally, a value can be determined from which the concentration can be inferred. Such a value may arise, for example, from the analysis of the generated image or images. Parameters such as contrast, brightness, morphology, colour, pattern or the like may provide a basis for this.

In a particular embodiment, the dispensing apparatus may have an actuating means. The actuating means can be used to actuate a section of the dispensing device to effect a discharge of the liquid sample, in particular the liquid drop, from the dispensing device. The actuating means can be a piezo-electric actuator. As a result, the dispensing device can be actuated straightforwardly. After dispensing liquid sample from the dispensing device, the liquid sample present in the fluid chamber flows into the discharge tubing.

The dispensing operation, in particular the dispensing of the liquid sample, can be carried out according to a drop-on-demand mode of operation. In this case, the dispensing apparatus provides a discrete and not a continuous sample discharge.

The control apparatus can regulate the cell concentration and/or particle concentration in such a way that it reaches the target value, or a value from a target value range that is or can be predetermined.

The dispensing apparatus can have a deflection and/or suction device. The diversion device is used to deflect the discharged liquid sample, in particular the discharged liquid drop. The suction device is used to suction off the dispensed liquid sample, in particular the discharged liquid drop. The discharged liquid sample can be diverted into a reject container and/or suctioned out. Alternatively, the discharged liquid sample can be fed into a container, in particular a container of the microtitre plate.

The diversion and/or suctioning can take place before the discharged liquid sample enters the container, in particular the container of the microtitre plate. In doing so, the discharged liquid sample can be diverted and/or suctioned out depending on the ascertained cell concentration and/or particle concentration. In particular, the discharged liquid sample can be diverted if the liquid sample contains no cells and/or no particles. Alternatively, the discharged liquid sample can be diverted and/or suctioned out if the number of cells and/or particles contained in the liquid sample is greater than a predetermined value, in particular greater than 1.

The dispensing apparatus can have a displacement device. The dispensing device and/or the container and/or the reject container can be displaced by means of the displacement device. The displacement operation can depend on the ascertained cell concentration and/or particle concentration and/or on a dosing operation. Thus, the liquid sample can be fed into the reject container if no cells, and/or no predetermined number of cells and/or particles, are present in the discharged liquid sample. By contrast, the discharged liquid sample can be fed into the container if a single cell and/or a single particle is arranged in the liquid sample.

The dispensing device, in turn, can be detachably connected to the remaining parts of the dispensing apparatus, in particular mechanically. As a result, the dispensing device can straightforwardly be switched out.

A computer program is particularly advantageous that comprises commands that, when the program is executed by a computer, cause the computer to carry out the method according to the disclosure. A data carrier on which the computer program according to the disclosure is stored is also advantageous. In addition, a data carrier signal that transmits a computer program according to the disclosure is advantageous.

### BRIEF DESCRIPTION OF THE DRAWING VIEWS

The subject matter of the disclosure is shown schematically in the figures, wherein elements that are the same or have the same effect are mostly provided with the same reference symbols. In the figures:

FIG. 1 shows a dispensing apparatus according to the disclosure in a state in which a drop of liquid is discharged,

FIG. 2 shows a dispensing device of the dispensing apparatus according to the disclosure, in a state in which the cell concentration in a discharge tubing of the dispensing device is too low,

FIG. 3 shows the dispensing device of the dispensing apparatus according to the disclosure, in a state in which the cell concentration in the discharge tubing is too low, after a portion of the liquid sample present in a fluid chamber has been suctioned into a tubing,

FIG. 4 shows the dispensing device of the dispensing apparatus according to the disclosure after dispensing the liquid sample that has been suctioned into the tubing,

FIG. 5 shows the dispensing device of the dispensing apparatus according to the disclosure, after a predetermined time has elapsed after the liquid sample has been suctioned into the tubing,

FIG. 6 shows the dispensing device of the dispensing apparatus according to the disclosure, after a portion of the liquid sample that has been suctioned in has been discharged from a tubing,

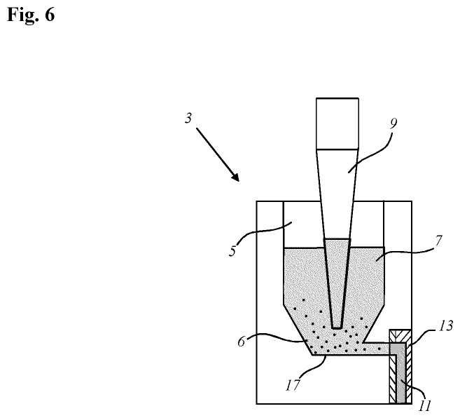

FIG. 7 shows the dispensing device of the dispensing apparatus according to the disclosure in a state in which the cell concentration in the discharge tubing is too high,

FIG. 8 shows the dispensing device of the dispensing device shown in FIG. 7 after a predetermined period of time.

### DETAILED DESCRIPTION

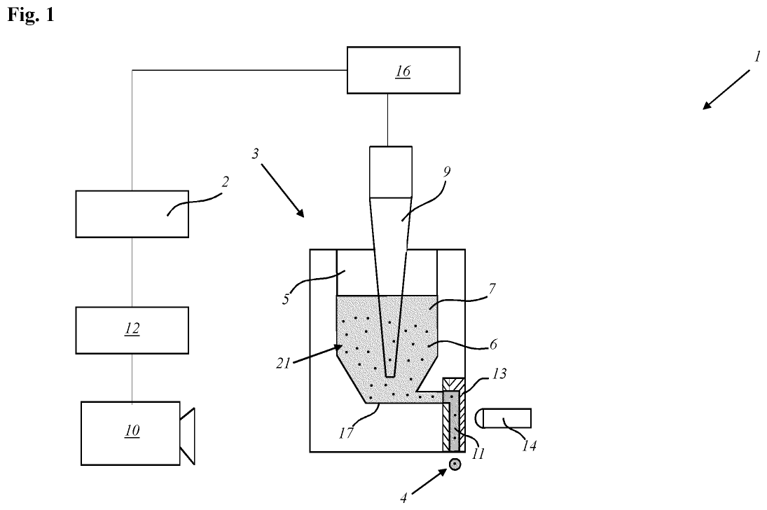

FIG. 1 shows: a dispensing apparatus 1 that has a dispensing device 3 for discharging a liquid drop 4, and a tubing 9 that has been inserted into the dispensing device 3. A liquid sample 21 is arranged in a part of the fluid chamber 5. The liquid sample 21 has a liquid 7 and cells 6. In the state shown in FIG. 1, the cells 6 are almost homogeneously distributed within the liquid 7 that is arranged in the fluid chamber 5.

Furthermore, the dispensing apparatus 1 has an imaging apparatus 10 for generating an optical image and an evaluation apparatus 12. The evaluation apparatus 12 is used to evaluate the generated image. In particular, by means of the evaluation apparatus 12 it can be ascertained whether the liquid drop 4 to be discharged, or liquid drop 4 that has been discharged from the dispensing device 3, has a cell 6. Alternatively or additionally, the number of cells arranged in the discharge tubing 11 or in a region of the discharge tubing 11 or in the discharged liquid sample 21, and thus the cell concentration, can be ascertained by means of the evaluation apparatus 12.

The dispensing apparatus 1 has a control apparatus 2 that is electrically connected to the evaluation apparatus 12 and is part of a computer that is not shown in greater detail. The control apparatus 2 is also electrically connected by means of a pressure or pump unit 16, which is fluidically connected to the tubing 9. By means of the pressure or pump unit 16, whether an overpressure or underpressure is present in the tubing 9 can be adjusted. Alternatively or additionally, the pressure or pump unit 16 can be designed in such a way that the tubing 9 is vented after the overpressure has been generated. To this end, the tubing 9 is fluidically connected to the environment.

The control apparatus 2 determines the cell concentration in the discharge tubing 11 or in the region of the discharge tubing 11 based on the information provided by the evaluation apparatus 12. In addition, the control apparatus 2 checks whether the cell concentration is less than or greater than a target value or equal to the target value. Depending on the result of this check, the pressure or pump unit 16 is controlled by the control apparatus 2 so as to apply an underpressure or an overpressure in the tubing 9.

The liquid drop 4 is discharged from the dispensing device 3 if the dispensing device 3 is actuated by means of an actuating means 14. This actuating means 14 may be a piezo-electric actuator that deforms a section 13 of the dispensing device 3 in order to discharge the liquid drop 4.

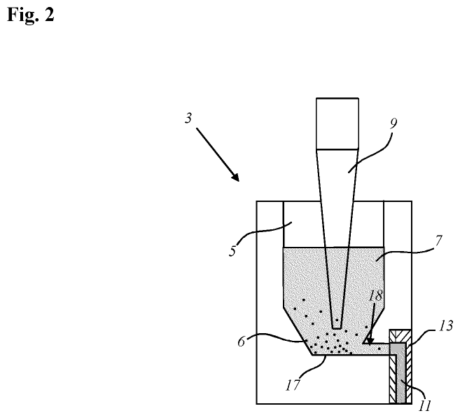

FIG. 2 shows the dispensing apparatus 1 according to the disclosure, in a state in which the cell concentration in the discharge tubing 11 of the dispensing device 3 is too low. This means that the control apparatus 2 has determined, based on the information provided by the evaluation apparatus 12, that the cell concentration in the region of the discharge tubing 11 or in the discharge tubing 11 is less than the target value.

This state can arise if a plurality of cells 6 accumulate at a bottom of a fluid chamber 17, as is apparent in FIG. 2. In this case, after a liquid drop 4 has been discharged from the dispensing device 3, a portion of the liquid sample 21 present in the fluid chamber 5 flows into the dispensing device 3. As is apparent from FIG. 2, the cell concentration of the liquid sample 21 in a region 18 of the fluid chamber 5 adjacent to the discharge tubing 11 is very low, such that a large number of dispensing operations tend to have to be performed in order for cells 6 to flow into the discharge tubing 11 together with liquid from the fluid chamber 5.

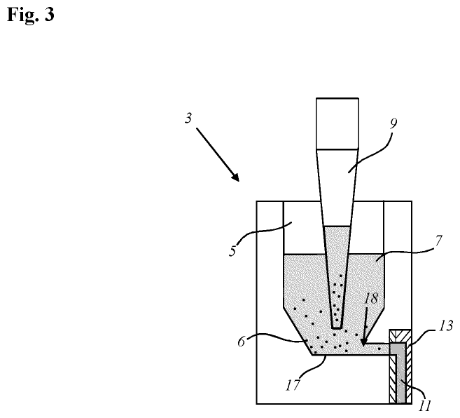

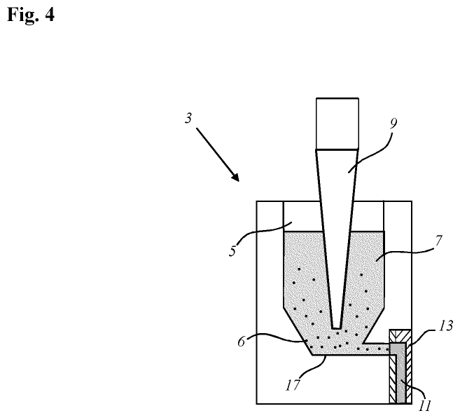

In order to rapidly increase the cell concentration in the discharge tubing 11, the control apparatus 2 causes the liquid sample 21 present in the fluid chamber 5 to be set in motion. This is described in greater detail with reference to FIGS. 3 and 4, in which agitation of the liquid sample 21 present in the fluid chamber 5 is achieved by mixing the liquid sample 21.

FIG. 3 shows the dispensing apparatus 1 in a state in which a portion of the liquid sample 21 present in the fluid chamber 5 has been suctioned into the tubing 9. In particular, an underpressure is applied in the tubing 9 by means of the pressure or pump unit 16, so that a portion of the liquid sample 21 present in the fluid chamber 5 is suctioned into the tubing 9. FIG. 4 shows the dispensing apparatus 1 in a state in which the portion of the liquid sample 21 suctioned into the tubing 9 in FIG. 3 has been discharged from the tubing 9 such that there is no longer any liquid sample 21 in the tubing 9. This can be realised by applying an overpressure in the tubing 9 by means of the pressure or pump unit 16.

The suctioning in of the liquid 7 into the tubing 9 and the discharge of the liquid 7 from the tubing 9 occur alternatingly. In addition, the suctioning in and out can be repeated multiple times in succession such that, as is apparent in FIG. 4, a more homogeneous distribution of the cells 6 in the fluid chamber 5 is achieved, in comparison to FIG. 3.

When a liquid drop 4 is discharged from the dispensing device 3, the region 18 of the fluid chamber 5 adjacent to the discharge tubing 11 has a higher cell concentration than is the case in the state shown in FIG. 3. Therefore, the probability is increased that cells 6 will flow into the discharge tubing 11 together with liquid after a dispensing operation. As a result, after one or more dispensing operations, the cell concentration increases in the discharge tubing 11 or in the region of the discharge tubing 11.

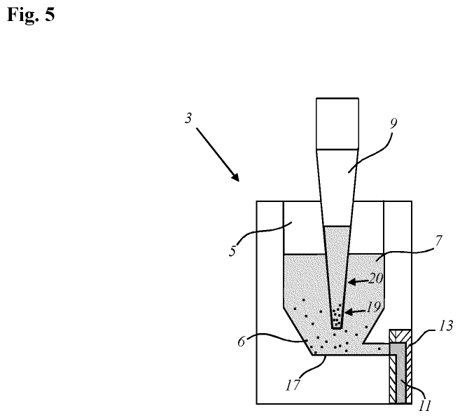

An agitation of the liquid 7 present in the fluid chamber 5, and/or an increase in the probability that liquid sample 21 having at least one cell will flow into the discharge tubing 11 after a dispensing operation, may additionally be realised by another method. This will be explained in greater detail with reference to FIGS. 3 and 5.

As described above, to realise the state shown in FIG. 3, a portion of the liquid sample 21 present in the fluid chamber 5 is suctioned into the tubing 9. In contrast to the process described with reference to FIG. 4, the suctioned-in portion of the liquid 7 is not discharged immediately after being suctioned in, but there is a delay of a predetermined length of time. The adjusted state after the time period has expired is shown in FIG. 5.

The portion of the liquid sample 21 suctioned into the tubing 9 has two liquid regions that differ from one another in cell concentration. In this case cells 6 that are present in the tubing 9 escape from the tubing 9 within the time period, such that the cell concentration and/or particle concentration increases in the liquid sample 21 that remains in the fluid chamber 5. In this case, a first liquid region 19 close to the bottom 17 of the fluid chamber has a higher cell concentration in the tubing 9 than a second liquid region 20. The second liquid region 20 is arranged within the tubing 9 above the first liquid region 19. The two liquid regions result from the fact that within the time period, the cells 6 sediment within the tubing 9 and therefore collect in the first liquid region 19 close to the bottom 17 of the fluid chamber.

After the predetermined time has expired, another portion of the liquid suctioned into the tubing 9 is discharged. The other portion is smaller than the portion suctioned into the tubing 9. In particular, the other portion discharged from the tubing 9 comprises the first liquid region 19 that has the high cell concentration. Because not all of the liquid 7 suctioned into the tubing 9 is discharged, but only the first liquid region 19 having the high cell concentration, the cell concentration in the remaining liquid sample 21 present in the fluid chamber 5 increases.

In addition, as a result of the liquid being suctioned in and discharged, a mixing of the liquid 7 in the fluid chamber 5 is achieved. In both cases it was advantageous that after a liquid drop 4 has been discharged from the dispensing device 3, cells 6 flow into the dispensing device 3 together with liquid 7, thus increasing the cell concentration in the discharge tubing 11. This occurs because more cells 6 are arranged in the region 18 of the fluid chamber 5 adjacent to the discharge tubing 11 than there were before the process was carried out, due to the above-described method.

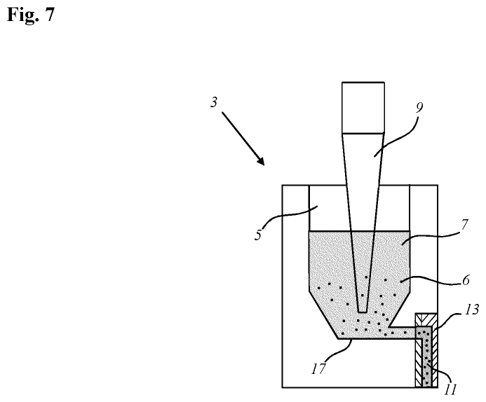

FIG. 7 shows the dispensing apparatus 1 in a state in which the cell concentration in the discharge tubing 11 and/or a region of the discharge tubing 11 is too high. Thus, the cell concentration in the discharge tubing 11 or in a region of the discharge tubing 11 is greater than the target value.

To reduce the cell concentration present in the discharge tubing 11, for a certain period of time no agitation takes place of the liquid sample 21 in the fluid chamber 5, i.e. the liquid sample 21 is not set in motion. This causes the cells 6 to sediment on the bottom 17 of the fluid chamber, which reduces the cell concentration in the region 18 of the fluid chamber 5 adjacent to the fluid dispenser 8.

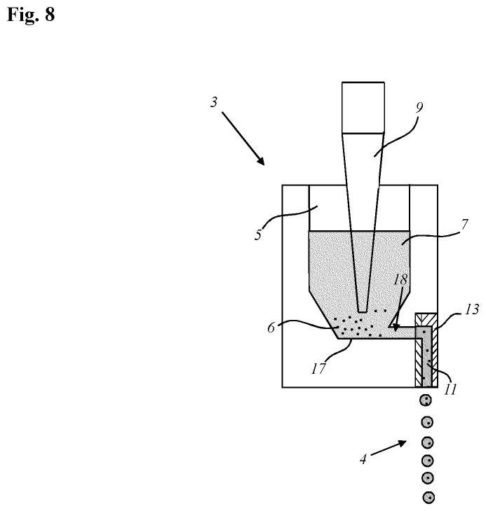

This state is shown in FIG. 8. In particular, in FIG. 8 it is apparent that a majority of the cells 6 present in the fluid chamber 5 are arranged at the bottom 17 of the fluid chamber.

To reduce the number of cells 6 present in the discharge tubing 11, a predetermined number of liquid drops 4 are then discharged, in particular after a predetermined period of time. The discharged liquid drops 4 are shown in FIG. 8 and each respectively have one or more cells. After each dispensing operation, a portion of the liquid sample 21 that is present in the region 18 adjacent to the discharge tubing 11 flows into the discharge tubing 11. Because the cell concentration of the liquid 7 flowing into the liquid dispenser 8 is low, the cell concentration in the discharge tubing 11 is also reduced as a result, as shown in FIG. 8. Thus, the cell concentration in the discharge tubing 11 is lower than in the state shown in FIG. 7.

### LIST OF REFERENCE SIGNS

1 Dispensing apparatus
2 Control apparatus
3 Dispensing device
4 Liquid drop
5 Fluid chamber
6 Cell
7 Liquid
9 Tubing
10 Imaging apparatus
11 Discharge tubing
12 Evaluation apparatus
13 Section of dispensing device
14 Actuating means
16 Pressure unit or pump unit
17 Fluid chamber base
18 Adjacent region
19 First liquid region
20 Second liquid region
21 Liquid sample

1. A method for setting a cell concentration and/or a particle concentration in a dispensing device (3) having a fluid chamber (5) into which a liquid sample (21) is introduced, the liquid sample (21) including a liquid (7) and cells (6) and/or particles, the method comprising:
determining the cell concentration and/or the particle concentration in one region of the dispensing device;
comparing the determined cell concentration and/or particle concentration with a target value; and
moving or not moving the cells and/or particles present in the fluid chamber (5) as a function of the result of the comparison.

2. The method according to claim 1, wherein the cells (6) and/or particles present in the fluid chamber (5) are moved if the determined cell concentration and/or particle concentration is less than the target value.

3. The method according to claim 1, wherein the cells (6) and/or particles present in the fluid chamber (5) are moved less strongly in a state in which the cell concentration and/or particle concentration is greater than the target value than in another state in which the cell concentration and/or particle concentration is less than the target value.

4. The method according to claim 1, wherein the cells (6) and/or particles are not moved if the determined cell concentration and/or particle concentration is greater than the target value or equal to the target value.

5. The method according to claim 1, further comprising dispensing liquid sample (21) from the dispensing device (3) after moving the cells (6) and/or particles, and/or after waiting a predetermined time after determining the cell concentration and/or the particle concentration.

6. (canceled)

7. The method according to claim 1, wherein moving the cells and/or particles present in the fluid chamber (5) comprises alternately suctioning a portion of the liquid sample (21) present in the fluid chamber (5) into a tubing (9) and discharging into the fluid chamber (5) at least a portion of the liquid sample (21) present in the tubing (9).

8. (canceled)

9. The method according to claim 7, wherein the portion of the liquid sample (21) suctioned into the tubing (9) is kept in the tubing (9) for a predetermined time, and after the predetermined time has elapsed, another portion of the suctioned-in liquid sample (21), which is smaller than the suctioned-in portion of the liquid sample (21), is discharged into the fluid chamber (5), and wherein the discharged other portion of the suctioned-in liquid sample (21) has a higher cell concentration and/or particle concentration than the portion of the liquid sample (21) that remains in the tubing (9).

10. (canceled)

11. (canceled)

12. (canceled)

13. The method according to claim 1, wherein determining the cell and/or particle concentration comprises ejecting a portion of the liquid sample (21) from the dispensing device (3), generating an image of the ejected portion of the liquid sample, and detecting cells and/or particles contained in the ejected portion of the liquid sample from the image.

14. (canceled)

15. The method according to claim 1, wherein determining the cell and/or particle concentration comprises generating an image of at least one region of a discharge tubing (11) of the dispensing device (3) in which a portion of the liquid sample (21) to be discharged is present and detecting cells and/or particles contained in the portion of the liquid sample from the image.

16. (canceled)

17. (canceled)

18. (canceled)

19. The method according to claim 1, further comprising
actuating a section (13) of the dispensing device to dispense a liquid drop (4) of liquid sample (21) from the dispensing device through a discharge tubing (11) of the dispensing device,
wherein liquid sample (21) flows from the fluid chamber (5) into the discharge tubing (11) after the liquid drop (4) is dispensed.

20. The method according to claim 1, further comprising dispensing liquid sample (21) from the dispensing device according to a drop-on-demand mode of operation.

21. (canceled)

22. The method according to claim 1, wherein the cell concentration and/or particle concentration is controlled such that the cell concentration and/or particle concentration reaches the target value or a value within a predetermined range of the target value.

23. A dispensing apparatus (1) comprising:
a dispensing device (3) including a fluid chamber (5) into which a liquid sample (21) is introduced, wherein the liquid sample (21) includes a liquid (7) and cells (6) and/or particles, the dispensing device (3) further including a mixing means (9, 16) for mixing the liquid sample (21) in the fluid chamber (5) to move the cells and/or particles;
an imaging apparatus (10) arranged to generate an image of a portion of the liquid sample (21) present in a region of the dispensing device (3) and/or in a liquid drop (4) of liquid sample (21) dispensed from the dispensing device (3);
an evaluation apparatus (12) configured to evaluate the image to ascertain information including whether the imaged portion of the liquid sample (21) has a cell (6) or a particle, and/or ascertain a number of cells or particles in the imaged portion of the liquid sample (21); and
a control apparatus (2) connected to the evaluation apparatus (12) and the mixing means;
wherein the control apparatus (2) or the evaluation apparatus (12) is configured to determine a cell concentration and/or a particle concentration in the imaged portion of the liquid sample (21) based on the information ascertained by the evaluation apparatus (12); and
wherein the control apparatus (2) is configured to compare the determined cell concentration and/or particle concentration with a target value, and operate or not operate the mixing means as a function of the result of the comparison.

24. (canceled)

25. The dispensing apparatus (1) according to claim 23, wherein the control apparatus (2) is configured to operate the mixing means to cause the cells (6) and/or particles to move if the determined cell concentration and/or particle concentration is less than the target value.

26. The dispensing apparatus (1) according to claim 23, wherein the control apparatus (2) is configured to operate the mixing means to cause the cells and/or particles to be moved less strongly in a state in which the cell concentration and/or the particle concentration is greater than the target value than in a different state in which the cell concentration and/or the particle concentration is less than the target value.

27. The dispensing apparatus according to claim 23, wherein the control apparatus (2) is configured to not operate the mixing means for a predetermined period of time if the determined cell concentration and/or particle concentration is greater than the target value or equal to the target value.

28. (canceled)

29. The dispensing apparatus (1) according to claim 23, wherein the mixing means comprises a tubing (9) in communication with the fluid chamber (5) and a pressure unit or pump unit (16) connected to the tubing (9) and to the control apparatus (2), the control apparatus (2) being configured to operate the pressure unit or pump unit (16) to suction a portion of the liquid sample (21) into the tubing (9) and cause the suctioned-in portion of the liquid sample (21) to be held in the tubing (9) for a predetermined period of time.

30. The dispensing apparatus (1) according to claim 23, wherein the dispensing device includes a discharge tubing (11) in flow communication with the fluid chamber (5), and the control apparatus (2) is configured to set the cell concentration and/or particle concentration in the discharge tubing (11) or a region of the discharge tubing (11).

31. (canceled)

32. (canceled)

33. The dispensing apparatus (1) according to, further comprising an actuating means (14) for actuating a section (13) of the dispensing device (3) for discharging liquid sample (21) from the dispensing device (3).

34. (canceled)

35. (canceled)

36. A non-transient computer readable storage medium comprising a computer program comprising commands that, when the computer program is executed by a computer, cause the computer to carry out the method according to claim 1.

37. (canceled)

38. (canceled)
